(19) United States
(12) Patent Application Publication
(10) Pub. No.: US 2006/0217589 A1
(43) Pub. Date: Sep. 28, 2006

(54) Pubovaginal sling implanter and procedure for the usage

(76) Inventors: Shaw P. Wan, Rolla, MO (US); Rosendo Martinez, O'Fallon, MO (US)

Correspondence Address: SHAW P. WAN, 594 SYCAMORE DRIVE, ROLLA, MO, 65401, US

(21) Appl. No.: 11/085,461

(22) Filed: Mar. 22, 2005

(51) Int. Cl. A61F 2/00

(52) U.S. Cl. 600/29

(57) ABSTRACT

A surgical instrument used for the implantation of a Pubovaginal Sling for treating female stress urinary incontinence comprising of a guide tube, a detachable tapered tube and a flexible shaft. These elements penetrates the body cavity, place the standard Pubovaginal Sling in the proper place and helps obtaining the proper tension of the Sling.

### BACKGROUND

1. Field of Invention

This invention relates to a surgical instrument used for the implantation of a Pubovaginal Sling for treating female stress urinary incontinence.

2. Description of Prior Art

Present techniques have shown the need for improved surgical instrumentation to minimize the risks of hemorrhage, injury to internal organs, sling erosion and postoperative urinary retention.

A literature review has shown the present techniques are associated with 5% of hemorrhagic complications, 4% bowel and bladder injury, 1% of graft erosion, and 3% of urinary retention.

Accordingly we have developed an instrument that would minimize the risks of the above-mentioned complications.

For many years, surgeons have used sling procedures to treat female urinary stress incontinence (SUI) in women. This procedure is referred to as the Pubovaginal Sling procedure. The procedure creates a support to the urethra and bladder neck complex. Providing this support, serves to increase the urethral closure pressure during stress to mitigate the involuntary loss of urine. In this procedure, a sling hanging from and secured to the pubo-abdominal side is used to support the urethra from below. There are many methods of implanting surgical slings for supporting the bladder neck. A couple of examples for attaching the sling to the pubic bone: May 2, 2000, Benderev, Naves & Legome, U.S. Pat. No. 6,056,688 and Apr. 25, 2000, Brenneman, Sauvageau & Gellman, U.S. Pat. No. 6,053,935.

Other procedures utilize a sling material with a plurality of holes that are sized to allow tissue ingrowths and increase the friction between the mesh and the tissue thus keeping the mesh in place. The implantation is accomplished by placing a plastic sheath over the mesh, penetrating a needle through the tissue for grasping the plastic sheath and mesh and pulling the plastic sheath and mesh together. After the plastic sheath is in place with the mesh then the plastic sheath is withdrawn leaving the mesh in place. Tissue damage results as the plastic sheath is pulled with the needle and the plastic sheath slides through the tissue. Implantation methods like Sep. 2, 2003, Staskin, Neisz & Westrum & Rocheleau, U.S. Pat. No. 6,612,977 uses a pair of needles to take the dilator in tow leaving the dilator dangling as it pulls the sling into place but does not provide the protection that a controllable, smooth guide tube provides to guide the sling into the proper place.

The main purpose of this invention is to provide a smooth, controllable passage of the sling material into the body cavity without sliding the sling material through the tissue or dangling any object and by providing a smooth transition for the sling material. This is accomplished by penetrating a substantially rigid guide tube with a penetrating tip through the tissue at the desired location. After the tube is in the desired passage, a flexible shaft brings the sling material inside the guide tube. When the sling material is in the desired location in relation with the tissue then the guide tube is pulled away from the tissue. This leaves the sling material inside the tissue in the desired place without the need to move the sling any more. Of course during the guide tube penetration there has to be some means of closing the guide tube-penetrating end. Purpose being, to prevent tissue damage while penetrating the tissue as well as having some means of gradual transition from the penetrating tip to the large perimeter required of the guide tube to accept the sling material inside the tube. A detachable tapered tube in combination with the penetrating tip of the flexible shaft protruding from the opening of the detachable tapered tube helps to penetrate the tissue and close the guide tube opening.

This instrument, consisting of a substantially rigid guide tube, a tapered tube and a flexible shaft, guided digitally as it is passed through the body. Its placement can be confirmed with cystoscopy. These features will minimize the risks of hemorrhaging as well as injury to the internal organs. During the procedure after the guide tube is in the proper place, the detachable tapered tube is removed from the guide tube and can be used as a spacer to avoid excessive pressure of the sling material on the bladder neck; therefore, reducing the risk of sling erosion and postoperative urinary retention.

During the development of current instrument, we discovered that modifications of our Nov. 3, 1998, U.S. Pat. No. 5,830,220 could be made to develop a new instrument that can achieve henceforth-mentioned goals.

To achieve safe passage of the instrument for implantation of the Pubovaginal Sling, it is better to enter suprapubically into the vagina.

The instrument requires a tapered end that would minimize injury to the tissue during its passage through the body. The internal diameter of the instrument however needs to be sufficiently large enough to accommodate the sling material, generally 5 mm or more. A means to pull the sling material into the body of the instrument for implantation is required.

We modified our U.S. Pat. No. 5,830,220 by adding a detachable tapered tube at the penetrating end of the instrument. The flexible shaft passes through the detachable tapered tube exposing the shaft's penetrating tip through the small opening of the detachable tapered tube. The detachable tapered tube creates a smooth transition between the flexible shaft's penetrating tip and the large perimeter of the guide tube. The flexible shaft has a suture tread holding capability to pull the sling inside the guide tube after the detachable tapered tube is removed.

We further modified our U.S. Pat. No. 5,830,220 by adding a (male/female) connection at the handle side of the instrument and in the griping means of the flexible shaft to prevent their disengagement during passage of the instrument through the body.

We also engineered a connection between the detachable tapered tube and the guide tube's penetrating end. This will prevent their disengagement during the passage through the body. It will not change the size internally or externally and does not require any twisting or snapping means during engagement.

The detachable tapered tube is designed of sufficient length so that it can be placed between the urethra and the sling material and used as a spacer toward the end of the implantation procedure. This would control the amount of pressure the sling material would put on the neck of the bladder.

Objects and Advantages

Accordingly, the main purpose of this invention is to provide a smooth passage of the sling material into the body that would minimize the amount of tissue damage and by not having to slide the sling material or to drag a dilator through the tissue cavity. This is accomplished by placing the guide tube with total control, at the desired location inside the tissue, bringing the sling material inside the guide tube and pulling the guide tube away from the tissue leaving the sling material inside the tissue in the desired place.

### BRIEF DESCRIPTION OF THE DRAWINGS

FIG. 1 is a perspective view of a Pubovaginal Sling Implanter of the present invention fully assembled.

FIG. 2 is a perspective view of a Pubovaginal Sling Implanter of the present invention as the separate portions are disconnected during the procedure.

FIG. 3 is a sectional view of the instrument of FIG. 1.

FIG. 4 is an exploded view of the instrument in FIG. 2.

FIG. 5 is a perspective view of another version of engagement method between the flexible shaft assembly and the guide tube of the present invention.

FIG. 6 is a perspective view of another version of engagement of the guide tube.

FIG. 7A is a perspective view of an eye type suture thread holder of the present invention.

FIG. 7B is a perspective view of a hook type suture thread holder of the present invention.

FIG. 8 is a perspective view of a different Pubovaginal Sling Implanter version of the present invention.

FIG. 9 is a perspective view of a portion of a different Pubovaginal Sling Implanter version of the present invention.

FIG. 10 is an exploded view of a different Pubovaginal Sling Implanter version of the present invention.

FIG. 11 is a perspective view of a portion of the different Pubovaginal Sling Implanter versions of the present invention.

FIG. 12 is a sectional view 12-12 of the instrument in FIG. 11.

FIG. 13 is a perspective view of another different Pubovaginal Sling Implanter version of the present invention.

FIG. 14 is a perspective view of a portion of the instrument in FIG. 13.

FIG. 15 is a sectional view of the instrument in FIG. 14.

FIG. 16 is a perspective view of another different Pubovaginal Sling Implanter version of the present invention.

FIG. 17 is a perspective view of a portion of the instrument in FIG. 16.

FIG. 18 is a sectional view of another different Pubovaginal Sling Implanter version of the present invention.

FIG. 19 is a view showing the preparation for performing the procedure using the Pubovaginal Sling Implanter.

FIG. 20 is a view showing the beginning of performing the procedure using the Pubovaginal Sling Implanter.

FIG. 21 is a view describing the initial implantation of the Pubovaginal sling.

FIG. 22 is a view describing the initial positioning of the Pubovaginal sling.

FIG. 23 is a view describing the final positioning of the Pubovaginal sling.

FIG. 24 is a view describing the adjustment procedure of the Pubovaginal sling.

FIG. 25 is a view describing the final implantation of the Pubovaginal sling.

Corresponding reference numerals throughout the several views of the drawings designates corresponding parts.

### DETAILED DESCRIPTION OF THE PREFERRED EMBODIMENT

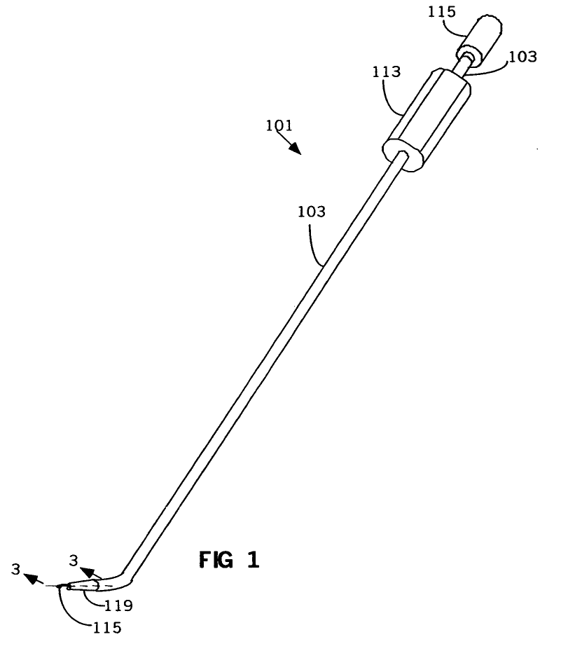

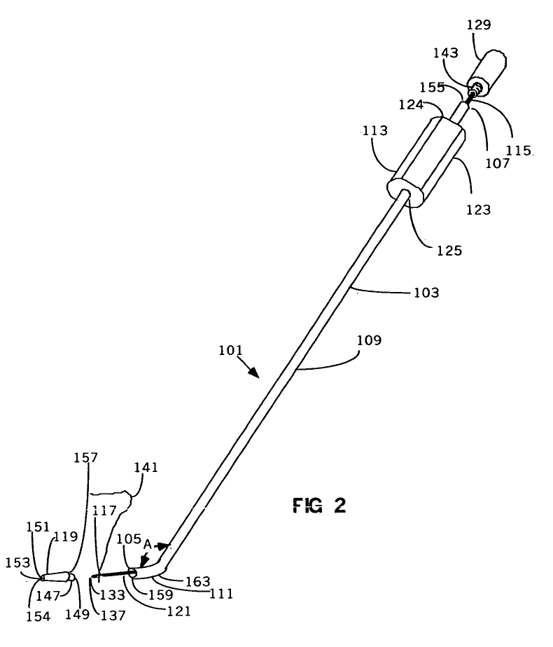

Referring now to the drawings, and first more particularly to FIGS. 1 and 2, a Pubovaginal Sling Implanter of the present invention is indicated in its entirety by the reference numeral 101. The surgical instrument 101 is used for the implantation of a Pubovaginal Sling for treating female stress urinary incontinence

As shown in FIG. 1, the instrument 101 comprises a substantially rigid guide tube 103, a handle assembly 113, a flexible shaft assembly 115 and a detachable tapered tube 119.

As shown in FIG. 2 and exploded FIG. 4, the instrument 101 comprises a substantially rigid guide tube 103 having opposite open ends 105 and 107, which are distal and proximal relative to a person using the instrument. The guide tube 103 has a stem portion 109 and a distal portion 111 extending generally laterally with respect to the stem portion 109 at an angle “A” not substantially less than 90 degrees. This angle is preferably 90-170 degrees and more preferably about 165 degrees. A handle assembly, indicated generally as 113, is fixedly attached to the stem portion 109 of the guide tube 103 generally adjacent the proximal open end 107 of guide tube 103. Proximal open end 107 of guide tube 103 has and internal thread 155 (not shown). The guide tube 103 is preferably, but not necessarily, formed from a single piece of bent metal tube.

As illustrated best in FIGS. 2 and 4, the handle assembly 113 comprises a generally straight handle portion 123. Handle assembly 113 has a central bore 125 there through for receiving the proximal end 107 of the guide tube 103 which is fixedly affixed to the handle in any suitable fashion. One manufacturing process for a metal instrument is for central bore 125 to be constructed slightly smaller than the perimeter of guide tube 103 at proximal end 107. During construction, guide tube 103 is cooled to very low temperatures and handle assembly 113 is heated to a very high temperature, thus expanding handle assembly 113 and contracting guide tube 103. With both components under those temperatures, guide tube 103 is slid into place inside central bore 125 of handle assembly 113 in the desired position. Then the components are let to stabilize to ambient temperature, thus contracting handle assembly 113 and expanding guide tube 103 to form a tight fit. If the components are made of other than metal, the handle assembly 113 can be made with central bore 125 slightly larger than the perimeter of guide tube 103 and the components bonded into place. The outer surface of handle 123 can be contoured of any shape, knurled and otherwise configured to provide a suitably comfortable and secure grip on the instrument 101.

Instrument 101 also includes a flexible shaft assembly 115 comprising of a stiff but flexible shaft 121 and grasping means 129. Flexible shaft 121 has a suture thread holder 117 at the distal end 133, a shaft middle portion 131 and a proximal end 135. Flexible shaft's proximal end 135 is attached permanently to distal end 145 of grasping means 129. Grasping means 129 has an external thread 143 at the distal end 145 of grasping means 129 to engage with internal thread 155 of proximal open end 107 of guide tube 103. This thread engagement is for the purpose of locking flexible shaft assembly 115 into place with guide tube 103.

The shaft 115 preferably comprises a stiff but flexible and resilient rod, which is substantially straight when unrestrained. The preferred shaft material is stainless steel or plastic (e.g., nylon 3/3) to keep costs sufficiently low to allow for disposal of flexible shaft assembly 115 after a single use. However, other materials may also be suitable. Distal end 133 of flexible shaft assembly 115 has a penetrating tip 137 to aid in the tissue penetration of the instrument. It is preferable for the penetrating tip 137 not to be sharp to prevent penetrating any blood vessels during the implantation procedure. The proximal end 135 of flexible shaft 121 may be secured to distal end 145 of grasping means 129 in any suitable fashion.

Flexible shaft assembly 115 is manually movable relative to guide tube 103 by pushing and pulling on grasping means 129. In this manner distal end part 133 of flexible shaft assembly 15 may be inserted into guide tube 103 and extracted away from guide tube 103.

Instrument 101 also includes detachable tapered tube 119 having open ends 149 and 151. Detachable tapered tube 119 as the name implies, is a tapered tube that the person using the instrument can install or remove from guide tube 103 during the implant procedure as required. Proximal large open end 149 of detachable tapered tube 119 has a shank 147 with the external perimeter of shank 147 constructed to match the inside shape of distal open end 105 of guide tube 103. Shank 147 must have the capability to slide inside distal open end 105 smoothly without any side play until it is stopped by shoulder 157 of detachable tapered tube 119 against rim 159 of distal end 105 of guide tube 103. Outside perimeter 150 of proximal open end 149 has the same perimeter size and shape as the outside perimeter of distal end 105 of guide tube 103. Small distal end 151 of detachable tapered tube 119 has an opening 153 with an inside diameter with just enough clearance to allow the exiting of flexible shaft 115.

During the implant procedure, shank 147 of proximal large open end 149 of detachable tapered tube 119 is inserted into distal open end 105 of guide tube 103. Shank 147 of detachable tapered tube 119 is slid toward the proximal end of guide tube 103. This penetration is stopped when shoulder 157 of detachable tapered tube 119 comes in contact with rim 159 of guide tube 103. Next distal end 133 of flexible shaft assembly 115 is inserted into the proximal open end 107 of guide tube 103. Flexible shaft 121 is slid inside guide tube 103 toward distal end 105 of guide tube 103. Flexible shaft assembly 115 continues going through the entire guide tube 103 and detachable tapered tube 119. This insertion is continued until external thread 143 of flexible shaft assembly 115 is engaged to the maximum thread travel into internal thread 155 of guide tube 103. Flexible shaft 121 has to be long enough for penetrating tip 137 of distal end 133 of flexible shaft assembly 115 is exposed through opening 153 of distal end 151 of detachable tapered tube 119. Penetrating tip 137 of distal end 133 of flexible shaft assembly 115 extends sufficiently beyond small distal end 151 of detachable tapered tube 119 to act as a penetrator. The exposure of penetrating tip 137 of distal end 133 of flexible shaft assembly 115 is limited for the purpose of distal end 133 to take a slight turn during the procedure. It is recommended that the distance from penetrating tip 137 to bend 163 of guide tube 103 be about 3 inches. Flexible shaft assembly 115 can be installed or removed from guide tube 103 by the person using the instrument during the implant procedure as required.

Suture thread holder 117 is located immediately adjacent penetrating tip 137 of distal end 133 of flexible shaft assembly 115. As shown in FIG. 7A, suture thread holder 117 comprises an opening 161 in the form of an eye next to penetrating tip 137 of flexible shaft 121 of flexible shaft assembly 115. It will be understood that other types of suture thread holders like hook 162, shown in FIG. 7B, could be used without departing from the scope of this invention. Other suture thread holding mechanisms commonly in existence could also be used without departing from the scope of this invention. The preferable suture thread holders is opening 161, which is sized to receive suture thread 141, much like the eye of a sewing needle.

While flexible shaft assembly 115 shown in the drawings is fabricated from multiple pieces, it will be understood that the shaft may be formed from a single piece of flexibly resilient rod. Other configurations are also possible.

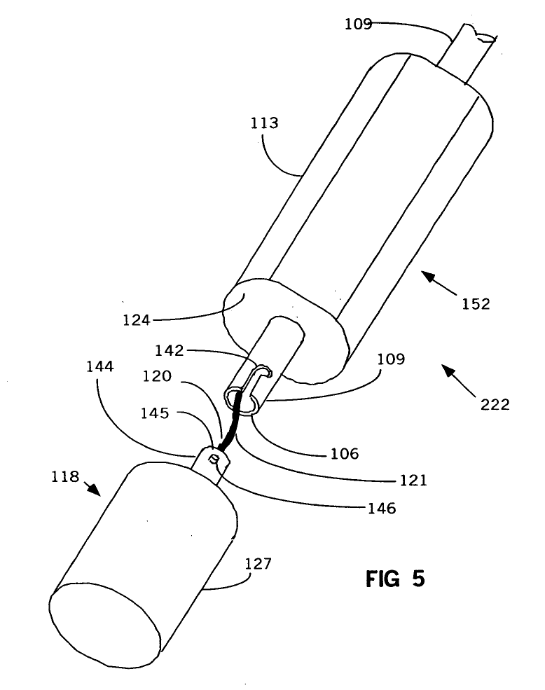

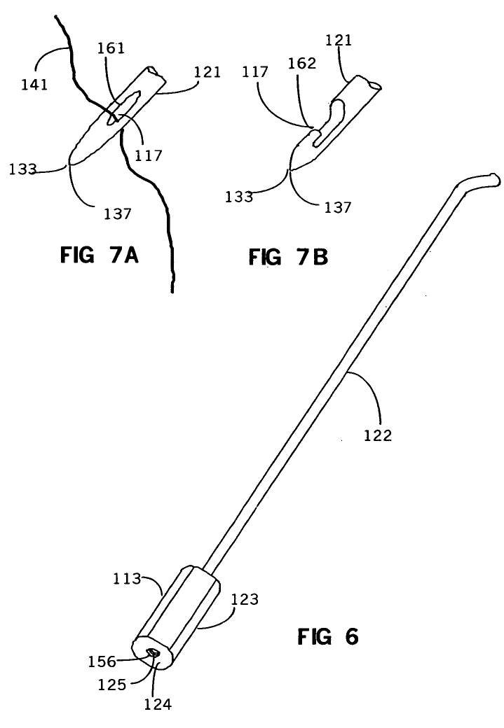

There are other variations of locking flexible shaft assembly 115 with guide tube 103 as shown in FIG. 5 and FIG. 6. As shown in FIG. 6, internal thread 155 of guide tube 103 does not have to be located at proximal end 107 of stem portion 109 as shown in FIG. 2. Stem portion 109 does not have to protrude through proximal end 124 of handle assembly 113 as shown in FIG. 2. Guide tube 122 is similar to guide tube 103 except it has internal thread 156 located at proximal end 124 of handle portion 123 as shown in FIG. 6, for engagement with external thread 143 of flexible shaft assembly 115.

Instrument 222 is another locking version of flexible shaft assembly 118 to guide tube 152, as shown in FIG. 5. In this version, proximal end 106 of stem portion 109 does not have any internal thread 155. Instead proximal end 106 of stem portion 109 can have a “J” shape slot 142 at the side of proximal end 106 of stem portion 109 of guide tube 152. In this version of the invention, flexible shaft assembly 118 does not have external thread 143 at distal end 145 of grasping means 127. For this version of attachment, grasping means 127 has a shank 144 attached coaxial to grasping means 127 at distal end 145. Shank 144 constructed with an external perimeter to match the inside shape of proximal open end 106 of guide tube 152. Shank 144 must have the capability to slide inside proximal open end 106 smoothly without any side play and to be partially spun inside stem portion 109.

Shank 144 has a pin 146 attached approximately mid way on one side of the perimeter of shank 144 with the axis of pin 146 oriented perpendicular to shank 144 axes. In this version of attaching engagement, distal end 133 of flexible shaft 121 of flexible shaft assembly 118 is penetrated at the proximal open end 106 of guide tube 152. The insertion continues all the way into guide tube 152. Just prior, shank 144 of distal end 145 of grasping means 127 comes in contact with proximal end 106 of guide tube 152, align pin 146 with “J” shape slot 142. When pin 146 reaches the end of travel inside. “J” shape slot 142, manually spin shaft assembly 118 to lock flexible shaft assembly 118 with guide tube 152. Of course the “J” shaped slot can be oriented during manufacturing for either left hand or right hand engagement.

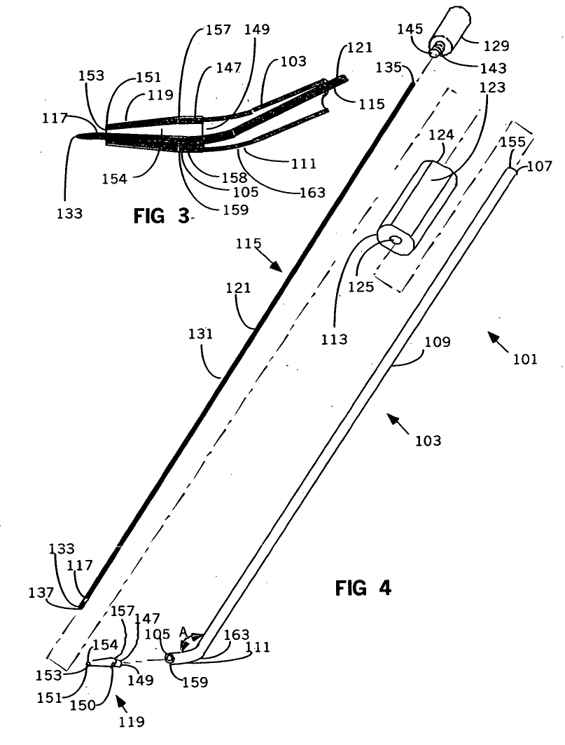

As one may have observed in FIGS. 2 and 4, detachable tapered tube 119 has no apparent locking means to keep detachable tapered tube 119 attached to distal end 105. It seems that detachable tapered tube 119 may come dislodged from guide tube 103 very easily but that is not the case. As shown in FIG. 3, shank 147 of large proximal end 149 of detachable tapered tube 119 is located next to bend 163 of guide tube 103. This location is very important as will be explained. When flexible shaft 121 of flexible shaft assembly 115 is not located inside bend 163 of guide tube 103, detachable tapered tube 119 can easily be remove or installed. However when the shank 147 of detachable tapered tube 119 is inserted into distal end 105 of guide tube 103 the insertion continues until shoulder 157 of detachable tapered tube 119 penetration is stopped by rim 159 of distal end 105 of guide tube 103. Next, flexible shaft assembly 115 is inserted into guide tube 103. The insertion is continued until external thread 143 and internal thread 155 are fully engaged. When flexible shaft 121 is in the region of distal portion 111, flexible shaft 121 has to take a turn inside bend 163 of guide tube 103. When flexible shaft 121 takes a turn inside bend 163 of guide tube 103, flexible shaft 121 tends to continue in a straight line. Therefore when flexible shaft 121 is inside the bend, the side of flexible shaft 121 exerts a pressure against inside passage 154 of detachable tapered tube 119. This pressure creates a friction between the side of flexible shaft 121 and the inside wall of passage 154 of detachable tapered tube 119. This pressure creates sufficient frictional force that requires a strong force to pull the detachable tapered tube 119 away from guide tube 103. To create an acceptable frictional force that prevents unintentional separation of the components, the following is recommended: Choose materials with high coefficient of friction like stainless steel over stainless steel. Other material combinations are also acceptable.

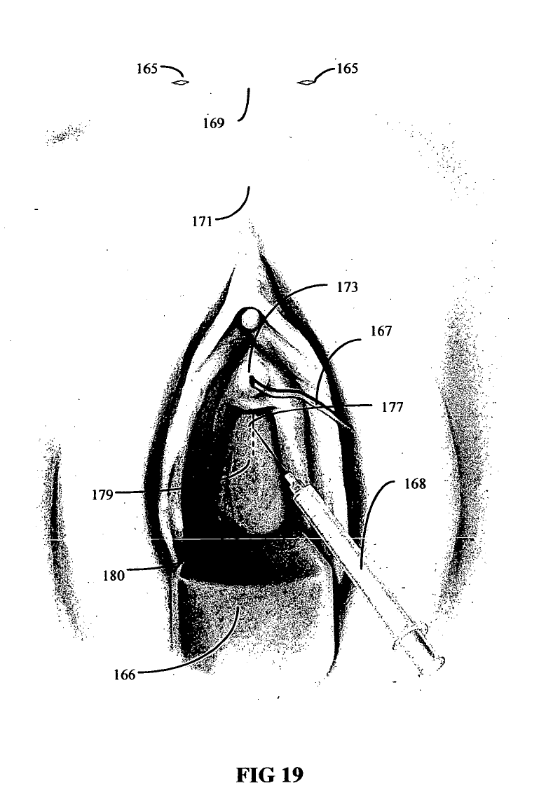

FIG. 19 through FIG. 25 presents a typical Pubovaginal Sling procedure using the Pubovaginal Sling Implanter described previously.

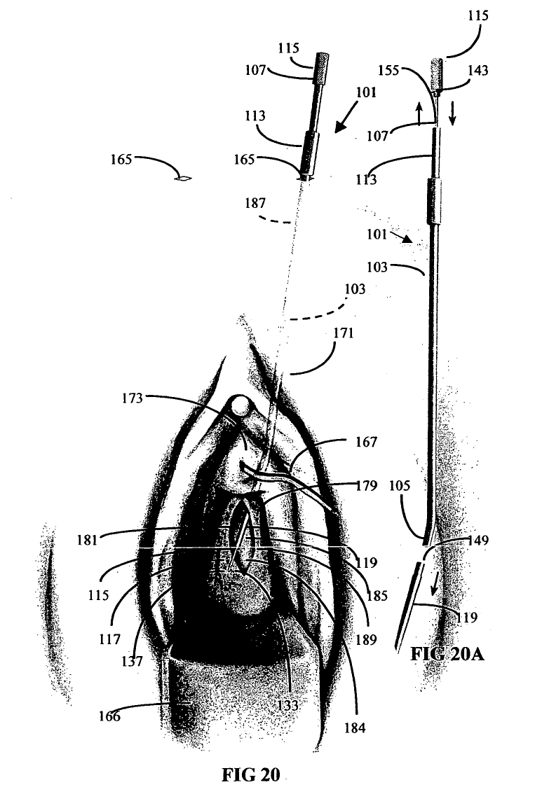

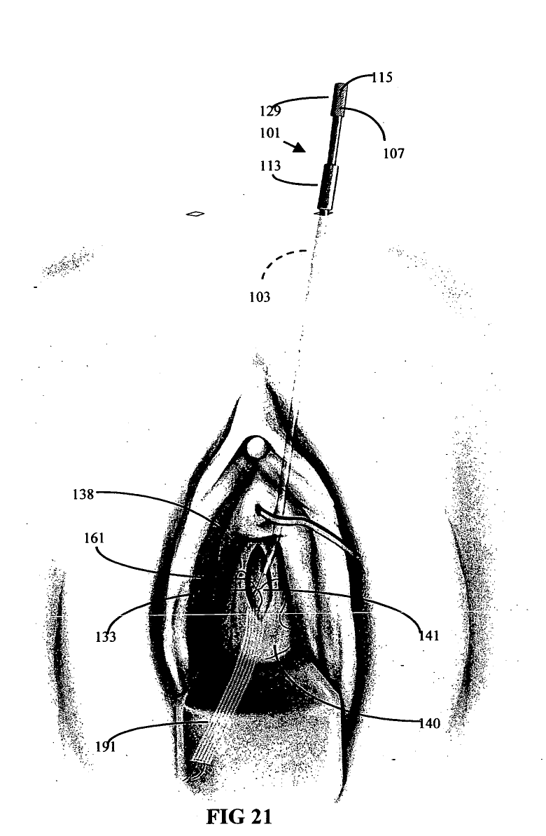

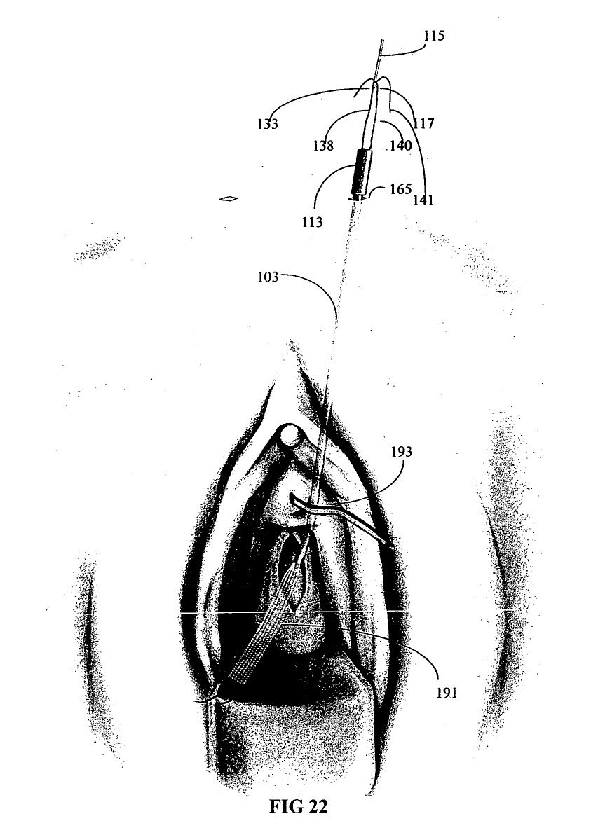

During the procedure after the patient is placed in the dorsal lithotomy position, under anesthesia, as shown in FIG. 19, standard retractor 166 is placed on lower vaginal opening 180 to facilitate the procedure. Then two small stab-wound incisions 165 about an inch apart are made on the center of female's lower abdomen 169 just above pubic symphysis 171. Subcutaneous tissue can be separated to the anterior rectus fascia with blunt dissection. Next, a Foley catheter 167 is placed in urethra 173. With Foley catheter 167 in place, urethra 173 and bladder neck 175 (not shown) can be identified by palpation. A two-centimeter skin marking 177 is made in anterior vagina 179 along urethra 173. The marked area is infiltrated with a local anesthetic containing epinephrine 168. Next as shown in FIG. 20, an incision is made in anterior vagina 179 following skin markings 177. Vagina mucosa 181 is separated from urethra 173 with combination of sharp and blunt dissection for a distance of about 1 to 2 centimeters. This separation must have enough space to allow the palpation of endopelvic fascia 185 to lateral urethra 173 with the surgeon's finger (not shown). Next, instrument 101 has to be assembled by inserting the large proximal end 149 of detachable tapered tube 119 into distal end 105 of guide tube 103. Followed by inserting flexible shaft assembly 115 into proximal end 107 of guide tube 103. The insertion is continued all the way until external threads 143 of flexible shaft assembly 115 and internal thread 155 of guide tube 103 are twisted for engagement. With one hand, the person performing the procedure grasps instrument 101 by handle assembly 113. Distal end 133 of flexible shaft assembly 115 is inserted into one of the suprapubic stab-wound incisions 165. Instrument 101 is thrust while penetrating tip 137 of suture thread holder 117 perforates through anterior rectus fascia 187. During the penetration, detachable tapered tube 119 helps expand the tissue. This expansion creates a smooth transition to the large perimeter of guide tube 103 as detachable tapered tube 119 and guide tube 103 slides along the posterior surface of pubic symphysis 171. With the surgeon's finger on endopelvic fascia 185 to the lateral urethra 173, the introducing penetrating tip 137 should be palpable by the surgeon's finger. With the finger, penetrating tip 137 is guided into vaginal incision 189 through the endopelvic fascia 185. The thrust of instrument 101 is continued until detachable tapered tube 119 clears vaginal incision 189. Flexible shaft assembly 115 is then unscrewed and partially drawn away from guide tube 103. This withdrawal continues until flexible shaft assembly 115 clears detachable tapered tube 119. When flexible shaft 115 is not in contact with detachable tapered tube 119, the pressure of the side of flexible shaft 121 against the inner wall of the detachable tapered tube 119 is released. Then detachable tapered tube 119 is removed from guide tube 103 as shown in FIG. 20A, by manually pulling detachable tapered tube 119 away from guide tube 103. Now flexible shaft assembly 115 is thrust again toward distal end 105 of guide tube 103 as shown in FIG. 20 A until suture thread holder 117 is exposed at the distal end of 105 of guide tube 103. Next, as shown in FIG. 21, a piece of standard sling material 191 is used and sling material 191 is cut into one-centimeter by ten centimeters in size. Now a long piece of suture thread 141 is inserted into one end of sling material 191. The suture thread has two ends, 138 and 140. Both ends 138 and 140 are inserted and passed through opening 161 of distal end portion 133 of flexible shaft assembly 115. Both ends of suture thread 141 are pulled until sling material 191 is adjacent to distal end 133 of flexible shaft assembly 115. The flexible shaft assembly 115 is withdrawn from guide tube 103. Flexible shaft assembly 115 is retracted further into guide tube 103 bringing along a section of sling material 191 into guide tube 103, and a section of suture thread 141 out of guide tube 103, as shown in FIG. 22.

Flexible shaft assembly 115 is continually pulled away from the patient until both suture ends of suture thread 141 exits suture thread holder 117 of flexible shaft assembly 115. At this time both ends of suture thread 141 are exposed outside guide tube 103.

While holding with the fingers the portion of sling material 191 that is exposed, guide tube 103 is withdrawn leaving sling material 191 at the desired location inside tissue 193. As guide tube 103 withdrawal is continued, sling material 191 is left compressed by the contracting tissue. The withdrawal of guide tube 103 is continued until guide tube 103 exits abdominal stab-wound incision 165, bringing along a section of suture thread 141. Guide tube 103 is now pulled away from abdominal stab-wound incision 165 until suture thread 141 exits guide tube 103.

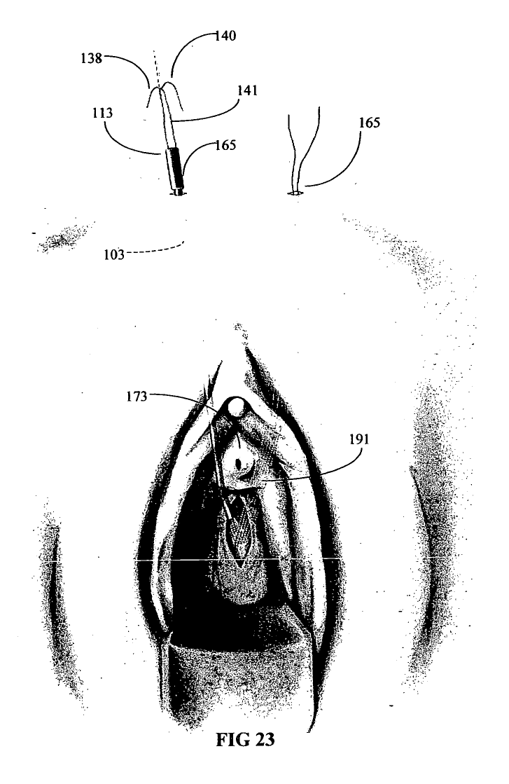

Detachable tapered tube 119 is again connected inside distal end 105 of guide tube 103 with flexible shaft assembly 115 protruding through detachable tapered tube 119. External threads 143 of flexible shaft assembly 115 and internal threads 155 of guide tube 103 are then locked into place. Instrument 101 is then inserted, the same way as previously explained, inside the other abdominal stab-wound incision 165. This time during the insertion, instrument 101 is guided on the other side of urethra 173. After detachable tapered tube 119 is removed, another suture thread 141 is inserted through the exposed end of sling material 191. Both suture thread ends 138 and 140 of suture thread 141 are inserted and passed through suture thread holder 117. Now both suture ends of suture thread 141 are pulled until sling material 191 is adjacent to suture thread holder 117 of flexible shaft assembly 115. Flexible shaft assembly 115 is then withdrawn further from the guide tube 103 bringing along the section of sling material 191 into guide tube 103 as shown in FIG. 23.

As flexible shaft assembly 115 is being withdrawn, detachable tapered tube 119 is then placed between looped sling material 191 and vaginal incision 189. Detachable tapered tube 119 is then placed crosswise across vaginal incision 189. This is accomplished to limit the travel of sling material 191 as sling material 191 begins to enter guide tube 103 as flexible shaft assembly 115 continues to be extracted from guide tube 103. This prevents excessive pressure of the sling material 191 on the bladder neck avoiding the risk of postop urinary retention or erosion of sling material 191 into urethra 173. The extraction of the flexible shaft is continued until the flexible shaft exits guide tube 103, the ends of suture thread 141 exits guide tube 103 and suture thread 141 exits suture thread holder 117.

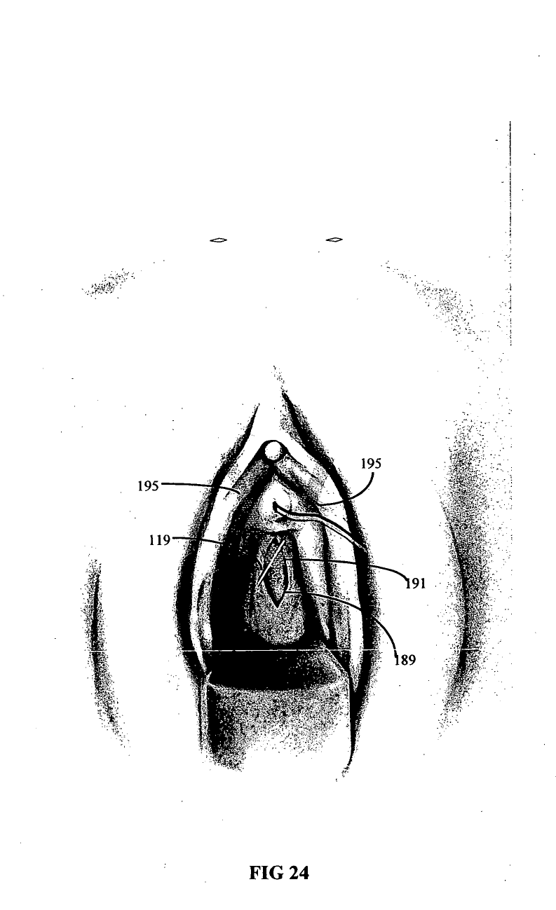

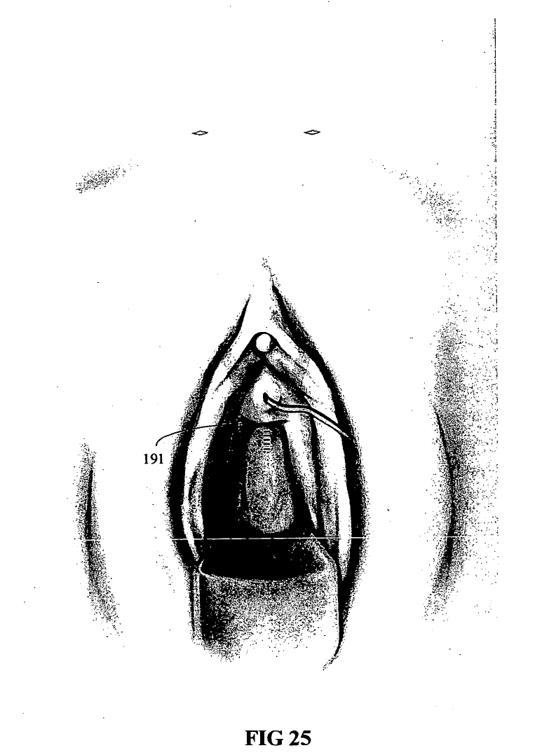

Manually suture thread ends 138 and 140 are pulled away from abdominal stab-wound incision 165 until sling material 191 is tight around detachable tapered tube 119 as shown in FIG. 24. Next, the withdrawal of guide tube 103 from stab-wound incision 165 commences. Guide tube 103 continues to be withdrawn from abdominal stab-wound incision 165 until guide tube 103 is outside abdominal stab-wound incision 165. Now suture thread 141 is pulled away from guide tube 103. Next, one of suture thread ends, 138 or 140 is pulled, thus sliding suture thread 141 away from sling material 191. Suture thread 141 continues to be pulled until the entire suture thread 141 clears abdominal stab-wound incision 165. The other suture thread 141 is also removed with the same method. Now the entire sling material 191 is implanted as shown in FIG. 25.

The procedure is almost completed. Now vaginal incision 189 is closed with absorbable suture thread (not shown) and lower abdominal stab-wound incisions 165 normally only require Steri-Strips (not shown). Foley catheter 167 and retractor 166 are now removed to complete the procedure. This procedure can be performed with a permanent sling such as a polypropylene mesh sling or a biodegradable sling such as cadaveric fascia, bovine or porcine collagen sling.

As with most inventions, there are other ways of accomplishing the same objective and still using the same basic principle and the Pubovaginal Sling Implanter is no exception. There are other versions of the Pubovaginal Sling Implanter. Looking at FIGS. 19 through 25, the main purpose of this invention is to provide a smooth passage of sling material 191 into tissue 193 with the minimal amount of tissue trauma and without sliding sling material 191 or a dilator through tissue cavity 193. This is accomplished by placing guide tube 103 at the desired location inside tissue 193, bringing sling material 191 inside guide tube 103 and pulling guide tube 103 away from tissue 193 leaving sling material 191 inside tissue 193 in the desired place without having to slide sling material 191 inside tissue 193 as shown in FIGS. 19 through 25. Of course there has to be some means of closing guide tube 103 and distal end 105 while penetrating tissue 193 as well as having some means of gradual transition from penetrating tip 137 to the large external cross sectional shape of guide tube 103. Detachable tapered tube 119 with penetrating tip 137 of flexible shaft 115 protruding from the opening of distal end 151 of detachable tapered tube 119 helps to penetrate tissue 193 and closes guide tube 103 opening.

Another version of our invention is installing any of the different kinds of detachable penetrator at distal end 105 of guide tube 103, which closes distal end 105 of guide tube 103 during the insertion of guide tube 103 into the tissue during the procedure. After guide tube 103 has penetrated through the tissue and the penetrator is exposed through the vagina opening, the detachable penetrator is removed to allow passage for the flexible shaft assembly.

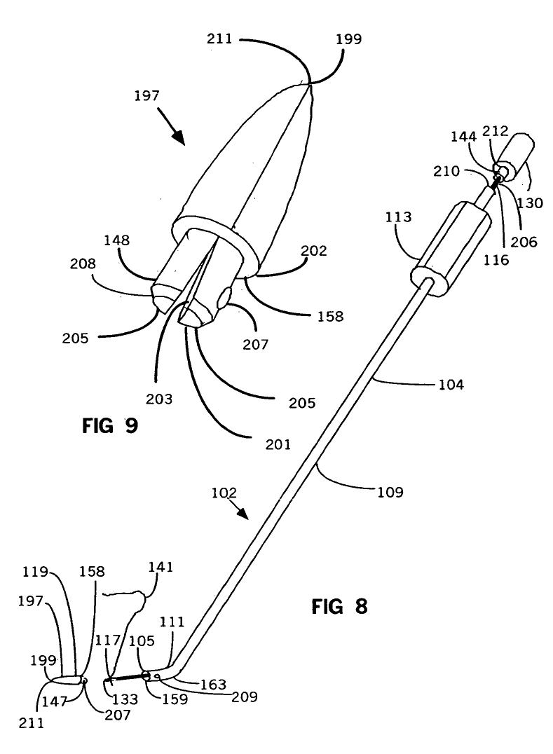

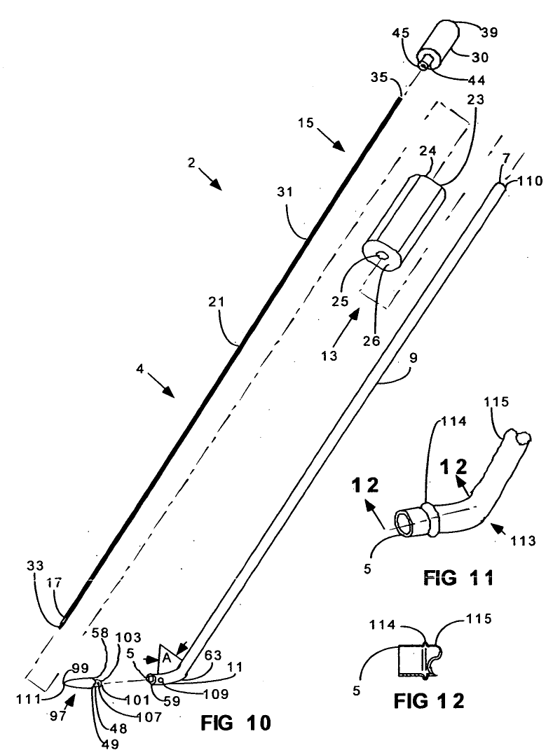

Instrument 102 is another method of accomplishing the same procedure as instrument 101 as shown in FIGS. 8 and 10. As shown in FIGS. 8 & 10, the main changes to instrument 101 to create instrument 102 is by substituting detachable tapered tube 119 of instrument 101 with a detachable penetrator 197. Also by making some minor modifications to guide tube 103, like removing internal thread 155 and adding a side opening 209 to distal end of guide tube 103 thus creating guide tube 104. Also by removing the external thread from the grasping means of the shaft assembly, creates the flexible shaft assembly 116 as described below.

As shown in FIGS. 8 & 10, the instrument 102 comprises a substantially rigid guide tube 104 having opposite open ends 105 and 206, which are distal and proximal relative to a person using the instrument. Guide tube 104 has a stem portion 109 and a distal portion 11 extending generally laterally with respect to stem portion 109 at an angle “A” not substantially less than 90 degrees. This angle is preferably 90-170 degrees and more preferably about 165 degrees. A handle assembly, indicated generally as 113, is fixedly attached to stem portion 109 of guide tube 104 generally adjacent proximal open end 206 of guide tube 104. Proximal open end 107 of guide tube 103 has and internal thread 155. (Notice that proximal open end 206 of guide tube 104 of instrument 102 does not require any internal thread like instrument 101).

As illustrated best in FIGS. 8 and 10, handle assembly 113 comprises a generally straight handle portion 123, preferably of metal, having a central bore 125 there through for receiving proximal end 206 of guide tube 104 which is fixedly affixed to the handle in any suitable fashion. Distal end 105 of guide tube 104 has a side opening 209 on the side of guide tube 104 very close to rim 159 of guide tube 104.

Instrument 102 also includes a flexible shaft assembly 116 comprising of a stiff but flexible shaft 121 and grasping means 130. Flexible shaft 121 has a suture thread holder 117 at distal end 133, a shaft middle portion 131 and a proximal end 135. Flexible shaft's proximal end 135 is attached permanently to distal end 145 of grasping means 130.

Distal end 145 of grasping means 130 has a shank 144 coaxial to grasping means 130 with the external perimeter constructed to match the inside shape of proximal end 206 of guide tube 104 and with the capability to slide inside proximal end 206 smoothly without side play and penetrate inside guide tube 104 until it is stopped by shoulder 212 of grasping means 130 of flexible shaft assembly 116 contacting rim 210 of guide tube 104. (Notice that grasping means 130 of flexible shaft assembly 116 does not require any external thread like distal end 145 of grasping means 129 of flexible shaft assembly 115 of instrument 161).

Flexible shaft assembly 116 preferably comprises a stiff but flexible and resilient rod, which is substantially straight when unrestrained. Flexible shaft assembly 116 is manually movable relative to guide tube 104 by pushing and pulling on grasping means 130. In this manner distal end part 133 of flexible shaft assembly 116 may be inserted into guide tube 104 and extracted away from guide tube 104.

Instrument 102 also includes a detachable penetrator 197 having a penetrating tip 199 at the distal end 211. Detachable penetrator 197 as the name implies, is a penetrator that the person using the instrument can install or removes from guide tube 104 during the implant procedure as required. When detachable penetrator 197 is attached to guide tube 104, flexible shaft assembly 116 can be inserted into guide tube 104 but it can only be inserted into guide tube 104 until distal end 133 of flexible shaft assembly 116 comes in contact with proximal end 201 of detachable penetrator 197. This version of the Pubovaginal Sling Implanter does not require having flexible shaft assembly 116 inside guide tube 104 when detachable penetrator 197 is locked with guide tube 104. Flexible shaft assembly 116 during insertion into guide tube 104 can only slide through the entire guide tube 104 when detachable penetrator 197 is not installed in guide tube 104. Flexible shaft assembly 116 can be installed or removed from guide tube 104 by the person using the instrument during the implant procedure as required.

Proximal end 201 of detachable penetrator 197 has a shank 148 that has a perimeter built to match the inside shape of distal open end 105 of guide tube 104 with the capability to slide inside distal open end 105 smoothly without any side play until it is stopped by shoulder 158 of detachable penetrator 197 against rim 159 of distal end 105 of guide tube 104. As shown in FIG. 9, proximal end 201 of detachable penetrator 197 has a split shank 203 running lengthwise from beginning of proximal end 201 of detachable penetrator 197 all the way until the split reaches shoulder 158 of detachable penetrator 197. This split forms two resilient legs 205 that are separated further at the proximal end of split 203 than at the distal end of split 203. Both resilient legs 205 have tapered ends 208 to facilitate the insertion of proximal end 201 into the opening of distal end 105 of guide tube 104, thus aiding the insertion of resilient legs 205 of detachable penetrator 197 into distal end 105 of guide tube 104. During resilient legs 205 insertion, both resilient legs 205 are forced toward each other and when shoulder 158 of detachable tape plug 197 comes in contact with rim 159 of guide tube 104, detachable tape plug 197 is spun by hand inside the opening of distal end 105 of guide tube 104 until dimple 207 that is located on the side of one of resilient legs 205 meets with side opening 209 of guide tube 104. At that moment, resilient legs 205 spring apart thus penetrating dimple 207 into side opening 209 of guide tube 104, locking detachable penetrator 197, inside guide tube 104 during instrument 102 penetration during the procedure. The large portion 202 of detachable penetrator 197 has the same shape and the same size perimeter as the perimeter of distal end 105 of guide tube 104 for a smooth transition inside the tissue.

Using instrument 102 for the implant procedure is similar to the implant procedure described with instrument 101 except instrument 102 has to be assembled by inserting split shank 148 of proximal end 201 of detachable penetrator 197 into distal end 105 of guide tube 104 before penetration into stab-wound incision 165. (Notice that when assembling instrument 102 in preparation for the insertion into tissue 193, flexible shaft assembly 116 is not installed). After detachable penetrator 197 clears the vaginal incision 189, detachable penetrator 197 is pulled away and removed from guide tube 104, and then distal end 133 of flexible shaft assembly 116 is inserted into proximal end 206 of guide tube 104 and penetrated all the way until the suture tread holder of flexible shaft assembly 116 is exposed through distal end 105 of guide tube 104. The remaining of the implant procedure described with instrument 101 is the same when the procedure is accomplished with instrument 102.

As previously mentioned, there are other versions of the Pubovaginal Sling Implanter like instrument 213, which is very similar to instrument 102. The flexible shaft assembly 116 of instrument 213 is identical to the flexible shaft assembly 116 of instrument 102. Detachable penetrator 197 of instrument 213 is identical to detachable penetrator 197 of instrument 102. The only difference between instrument 213 and instrument 102 is that guide tube 104 is replaced by guide tube 215.

As shown in FIGS. 10, 11 and 12, guide tube 215 is very similar to guide tube 104 except guide tube 215 does not have a side opening 209 for locking detachable penetrator 197 to guide tube 104 but instead it has an internal swage groove 214 next to distal end 105 of guide tube 215 for locking with dimple 207 of detachable penetrator 197.

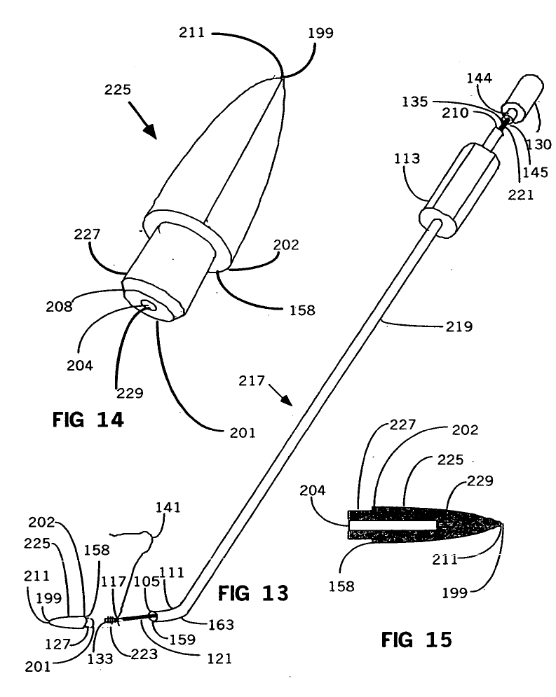

Another version of our invention is instrument 217. Instrument 217 as shown in FIGS. 13, 14 and 15, is very similar to instrument 102. Guide tube 219 is similar to guide tube 104 except side opening 209 is not required. Guide tube 104 will work with instrument 217 without any modification. Distal open end 105 must be circular, not like the other guide tubes, which can be of any shape as long it matches the size and shape of the shank of the detachable penetrator during the insertion into the tissue. Flexible shaft assembly 221 is similar to flexible shaft assembly 116 except flexible shaft 221 at distal open end 133 has external thread 223 next to suture thread holder 117.

Detachable penetrator 225 as shown in FIG. 14, is different than detachable penetrator 197 as shown in FIG. 9. As shown in FIG. 13, instrument 217 also includes detachable penetrator 225 having a penetrating tip 199 at distal end 211. Detachable penetrator 225 as the name implies, is a detachable penetrator that can be installed or removed from guide tube 219 during the implant procedure as required.

Proximal end 201 of detachable penetrator 225 has shank 227 with a circular perimeter built to match the size of the inside of distal open end 105 of guide tube 219 with the capability to slide inside distal open end 105 smoothly without any side play until it is stopped by shoulder 158 of detachable tapered plug 225 against the rim 159 of distal end 105 of guide tube 219. As shown in FIGS. 13, 14 and 15, proximal end 201 of detachable penetrator 225 has counterbore 204 and an internal thread 229 to engage with external thread 223 of distal end 133 of flexible shaft 221 during the instrument 217 penetrations and detachable penetrator 225 is unscrewed from flexible shaft 221 after instrument 217 has penetrated tissue 193 during the procedure as shown in FIG. 21 and detachable penetrator 225 is exposed in preparation to accept sling material 191 by suture thread holder 117 of flexible shaft 221.

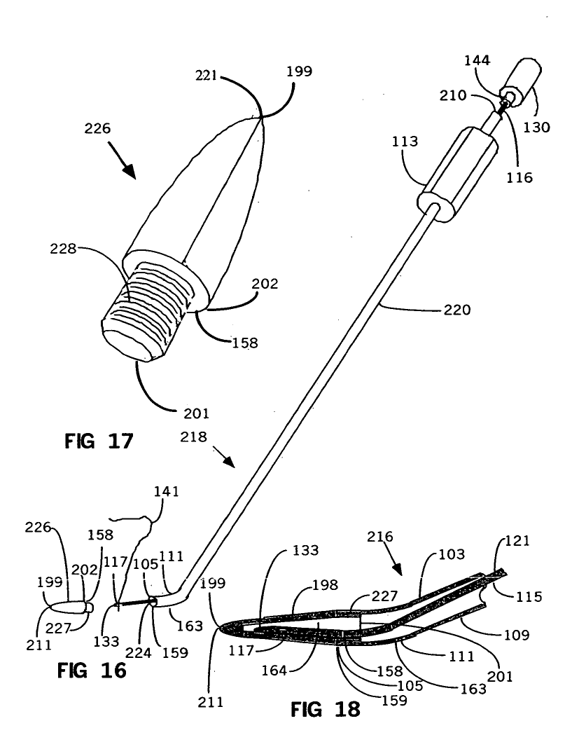

Another version of our invention is instrument 218. Instrument 218 is similar to instrument 217 except instrument 218 has an internal thread 224 at distal end 105 of guide tube 220 to accept a detachable penetrator 226 which has external threads 228 at proximal end 201 as shown in FIGS. 16 and 17.

Another version of our invention is instrument 216. Instrument 216 is similar to instrument 101 except instrument 216 uses detachable penetrator 198 instead of detachable tapered tube 119. Instrument 216 includes a detachable penetrator 198 having a penetrating tip 199 at distal end 211. The detachable penetrator 198 as the name implies, is a detachable penetrator that can be installed or removed from guide tube 103 during the implant procedure as required.

Proximal end 201 of the detachable penetrator 198 has shank 227 with a circular perimeter built to match the size of the inside of distal open end 105 of guide tube 109 with the capability to slide inside distal open end 105 smoothly without any side play until it is stopped by shoulder 158 of detachable penetrator 198 against rim 159 of distal end 105 of guide tube 103. As shown in FIG. 18, proximal end 201 of detachable penetrator 198 has a cavity 164 to allow distal end 133 of flexible shaft assembly 115 inside cavity 164 when flexible shaft assembly 115 is as far as possible into guide tube 103 and external thread 143 of flexible shaft 115 is engaged to internal tread 155 of guide tube 103.

Just like detachable tapered tube 119 has no apparent locking means to keep detachable tapered tube 119 attached to distal end 105 and it seems that detachable tapered tube 119 may come dislodged from guide tube 103 very easily but that is not the case. The same is true with detachable penetrator 198. As shown in FIG. 18, shank 227 of proximal end 201 of detachable penetrator 198 is located next to bend 163 of guide tube 103. This location is also very important just like detachable tapered tube 119. When flexible shaft 121 of flexible shaft assembly 115 is not located inside bend 163 of guide tube 103, the detachable penetrator 198 can easily be removed or installed. However when the shank 227 of detachable penetrator 198 is inserted into distal end 105 of guide tube 103 until shoulder 157 of detachable penetrator 198 penetration is stopped by rim 159 of distant end 105 of guide tube 103 and the flexible shaft assembly 115 is inserted into guide tube 103 until external thread 143 and internal thread 155 are fully engaged. Flexible shaft 121 has to take a turn inside bend 163 of guide tube 103. When flexible shaft 121 takes a turn inside bend 163 of guide tube 103, flexible shaft 121 tends to continue in a straight line therefore side of flexible shaft 121 exerts a pressure against inside passage 164 of detachable penetrator 198 as flexible shaft 121 takes the turn. This pressure creates a friction between the side of flexible shaft 121 and the inside wall of cavity 164 of detachable penetrator 198. This pressure creates sufficient frictional force that requires a strong force to pull detachable penetrator 198 away from guide tube 103.

Small distal end 151 of detachable tapered tube 103 can be truncated or wedge shaped at the tip to aid slicing through the tissue. It can also have the shape of a frustum of a cone that is shaped like a mechanical pencil without the lead or have the shape of a pyramid or any kind of shape that will help spread the tissue over the wall thickness of the tube as the instrument penetrates through the tissue. Almost any kind of shape will work.

In view of the above, it will be seen that the several objects of the invention are achieved and other advantageous results attained. As various changes could be made in the above constructions without departing from the scope of the invention, it is intended that all matter contained in the above description or shown in the accompanying drawings shall be interpreted as illustrative and not in a limiting sense.

For purposes of illustration only, guide tube 103 may be formed from a length of stainless steel tubing having an inside diameter of 0.17 in. and an outside diameter of 0.21 in. Stem 109 has a length of about 9.4 inches and distal sections 111 a length of about 0.4 inch with an angle of about 165 degrees between each other. The detachable tapered tube 119 is about 1.9 inches long, 0.2 inches of outside diameter at one end and 0.14-inch diameter at the other end. The internal hole is about 0.12 inch at one end and 0.06 inch at the other end. Flexible shaft 121 of flexible shaft assembly 115 has a diameter of about 0.06 inches and it is about 12.1 inch long. Suture thread holder 117 of flexible shaft assembly 115 is an elongated hole about 0.12 inches long by 0.02 inches wide with a radius at both ends. Grasping means 129 of flexible shaft assembly 115 has a diameter of about 0.28 inches by 0.81 inches long. Handle assembly 113 of guide tube 103 has a across section of 0.28 inch by 0.38 inch and about 1.1 inch long. All components are made of stainless steel.

### SUMMARY OF THE INVENTION

The object of this invention is to provide an instrument for use in positioning a Pubovaginal Sling that creates a support to the urethra and bladder neck complex with minimum tissue injury; the provision of such an instrument which facilitates guiding a Pubovaginal Sling in the perfect position; which is safe and easy to use, and which can be designed for either left-hand or right-hand use; and the provision of such an instrument which is simple in design for economical manufacture.

Briefly, an instrument of the present invention comprises a substantially rigid guide tube having opposite open ends, which are distal and proximal relative to a person using the instrument. The guide tube has a stem portion and a distal end portion extending generally laterally with respect to the stem portion. A handle is provided on the stem portion of the guide tube generally adjacent the proximal end of the guide tube. A stiff but flexible shaft inside the guide tube has a distal end part movable distally relative to the guide tube.

A detachable tapered tube having open ends. The large proximal end is partially inserted inside the distal end of the rigid guide tube with means to prevent the further penetration of the detachable tapered tube into the guide tube. The smaller distal end of the detachable tapered tube has an inside diameter with just enough clearance to allow the exiting of said flexible shaft. A suture thread holder with a penetrating tip for penetrating tissue is provided on the distal end part of the flexible shaft. It extends through and beyond the open distal end of the detachable tapered tube. The Flexible shaft has grasping means at the proximal end externally to the guide tube. The flexible shaft's perimeter of the instrument is considerably smaller than the internal path of the guide tube. This allows the sliding of the flexible shaft around the bend in the guide tube. The guide tube's passage is large enough to allow a Pubovaginal Sling to slide inside the tube without any difficulty. The shaft is manually movable relative to the guide tube. The distal end part of the shaft can be moved from an extended position in which the suture thread holder is generally adjacent the open distal end of the detachable tapered tube, to a retracted position in which the suture thread holder is bringing into the guide tube the Pubovaginal Sling. The distal end portion of the guide tube is configured to guide the distal end part of the shaft along a substantially straight path. It extends generally laterally at an angle of not substantially less than ninety degrees with respect to the stem portion of the guide tube.

Internal threads are located in the proximal end of the guide tube to engage with external threads on the proximal end of the flexible shaft. This locks the shaft in place inside the guide tube at the start of the procedure. The purpose of the removable detachable tapered tube is for narrowing the passage of the flexible shaft exit. This prevents human tissue from entering the tube's cavity during the penetration of the instrument into the tissue. It also provides a large opening for the guide tube, which allows the insertion of a Pubovaginal Sling-into the guide tube when the detachable tapered tube is removed.

The detachable tapered tube is kept in place in the distal end of the guide tube by the friction between the side of the flexible shaft and the inner wall of the detachable tapered tube. The load to create the friction between the side of the flexible shaft and the inner wall of the detachable tapered tube is developed by the stiff flexible shaft going through the bend just before the opening at the distal end of the guide tube. This forces the side of the flexible shaft against the inner wall of the detachable tapered tube, therefore providing a tight fit.

During the procedure the patient is placed in the dorsal lithotomy position. The procedure can be done with general, regional or even local anesthesia. After the patient is properly prepped and draped, two small stab-wound incisions about an inch apart are made on the center of the female's lower abdomen just above the pubic symphysis. The subcutaneous tissue can be separated to the anterior rectus fascia with blunt dissection. Next, a Foley catheter is placed in the urethra. With the catheter in place the urethra and the bladder neck can be identified by palpation. A two-centimeter marking is made in the anterior vagina along the urethra. The marked area is infiltrated with a local anesthetic containing epinephrine. Next, an incision is made in the anterior vagina following the skin markings. The vagina mucosa is separated from the urethra with combination of sharp and blunt dissection for a distance of about 1 to 2 centimeters with enough space created to allow the palpation of the endopelvic fascia to the lateral urethra with the surgeon's finger. Next, the instrument has to be assembled by inserting the detachable tapered tube's large proximal end into the guide tube distal end followed by inserting the flexible shaft into the guide tube proximal end all the way until the threads of the flexible shaft and the thread of the guide tube are twisted for engagement. With one hand the person performing the procedure grasps the instrument by the handle. The distal end of the flexible shaft is inserted into one of the incisions of the suprapubic stab-wounds thrusting the instrument while the penetrating tip of the suture thread holder perforates through the anterior rectus fascia. The large proximal end of the removable tapered tube helps expand the tissue creating a smooth transition to the large perimeter of the guide tube as the detachable tapered tube and the guide tube slides along the posterior surface of the pubic symphysis. With the surgeon's finger on the endopelvic fascia to the lateral urethra, the introducing penetrating tip should be palpable by the surgeon's finger and the penetrating tip is guided into the vaginal incision through the endopelvic fascia. The thrust of the instrument is continued until the detachable tapered tube clears the vaginal incision. The shaft is unscrewed and partially drawn away from the guide tube until the shaft clears the detachable tapered tube. This releases the pressure of the side of the flexible shaft against the inner wall of the detachable tapered tube. The detachable tapered tube is removed from the guide tube and the flexible shaft is thrust again toward the distal end of the guide tube until a suture thread holder is exposed at the distal end of the guide tube. Next, a piece of standard sling material is used and the sling is cut into one-centimeter by ten centimeters in size.

Now a long piece of suture thread is inserted into one end of the sling material. Both ends of the suture thread are then inserted and passed through the opening at the distal end portion of the flexible shaft and the suture thread is pulled until the sling material is adjacent to the distal end of the flexible shaft.

The flexible shaft is pulled away from the guide tube, bringing with it a section of the sling material and a section of the suture thread. After the desired penetration of the sling material is made inside the guide tube, the flexible shaft is grabbed with one hand while keeping the tension on the exposed sling material. The flexible shaft is withdrawn while maintaining the tension on the sling material. The flexible shaft is continued to be withdrawn from the guide tube bringing the suture thread along with the flexible shaft. The withdrawal is continued until the flexible shaft is away from the guide tube.

The flexible shaft continues to be pulled away from the patient until the ends of the suture thread exits the flexible shaft opening. While holding with the fingers the portion of the sling that is exposed, the guide tube is withdrawn leaving the sling material at the desired location inside the tissue. The sling material is held in place by the contracting tissue and the guide tube is continuing to be pulled until it is completely away from the abdominal incision and the suture thread exits the guide tube with the suture thread left exposed from the abdominal incision. The detachable tapered tube is again connected inside the distal end of the guide tube and then again the flexible shaft is inserted into the proximal end of the guide tube completely until the flexible shaft protrudes through the detachable tapered tube. The threads of the flexible shaft and the guide tube will lock into place. The instrument is then inserted the same way as previously explained inside the other abdominal incision.

This time the instrument is guided on the other side of the urethra and the bladder neck. After the detachable tapered tube is removed, another suture thread is inserted through the exposed end of the sling material. Then both ends of the suture thread are inserted and passed through the opening at the distal end portion of the flexible shaft. The suture thread is pulled until the sling material is adjacent to the distal end of the shaft. The flexible shaft is withdrawn from the guide tube. The flexible shaft is retracted further into the guide tube bringing along a section of the sling material. As the flexible shaft is withdrawn, the detachable tapered tube is placed through the looped suture thread. The sling material and the detachable tapered tube is then placed crosswise across the vaginal incision to limit the travel of the sling material as the flexible shaft continues to be extracted from the guide tube as the sling material begins to enter the guide tube. This prevents excessive pressure of the sling material on the bladder neck; therefore, avoiding the risk of postop urinary retention or erosion of the sling into the urethra.

After the sling material is tight around the detachable tapered tube, the flexible shaft continues to be withdrawn from the guide tube bringing the suture thread through the opening of the proximal end of the guide tube. The flex shaft continues to be withdrawn from the guide tube until the suture thread is released from the flexible shaft leaving the suture thread exposed at the proximal end opening of the guide tube. The guide tube is then pulled away from the tissue leaving the sling material in place looped from two tissue openings and preventing any further travel by the looped sling material around the detachable tapered tube. The procedure is almost completed.

Now one end of the exposed suture threads is pulled until the entire suture thread is away from the incision. The same process is repeated with the other suture thread. The detachable tapered tube is now removed from the area leaving the entire sling implanted. The vaginal incision is closed with absorbable suture threads where as the lower abdominal incisions normally only require Steri-Strips. The catheter is now removed to complete the procedure. This procedure can be performed with a permanent sling or a biodegradable sling such as cadaveric fascia, bovine or porcine collagen sling.

| | | |
|---|---|---|
| 101 | Instrument | With taper tube |
| 102 | Instrument | Instrument with Detachable penetrator with side hole in guided tube |
| 103 | Guided tube | Internal thread guide tube at proximal end |
| 104 | Guided tube | With side hole at distal end |
| 105 | Distal end | of Guided tube 103 |
| 106 | Proximal end | Proximal end of “J” slot engagement locking version |
| 107 | Proximal end | of Guided tube 103 |
| 108 | | |
| 109 | Stem | of all guied tubes |
| 110 | | |
| 111 | Distal portion | of all guied tubes |
| 112 | | |
| 113 | Handle assembly | Handle assembly of all guided tubes |
| 114 | | |
| 115 | Flexible shaft assembly | Flexible shaft assy with threaded distal end of grasping means |
| 116 | Flexible shaft assembly | Flexible shaft assy plain (no threads & no pin) |
| 117 | Suture thread holder | for all flexible shaft assembly |
| 118 | Flexible shaft assembly | Flexible shaft assy with pin engaging “J” shape slot |
| 119 | Detachable Taper Tube | |
| 120 | | |
| 121 | Flexible shaft | Flexible shaft the actual flexible rod of all flexible shaft assembly |
| 122 | Guided tube | Internal threads at proximal end of handle assy |
| 123 | Handle portion | Handle portion of guide tube |
| 124 | Proximal end | Proximal end of handle portion of all guided tubes |
| 125 | Central bore | Central bore of handle portion of all guided tubes |
| 126 | | |
| 127 | Grasping means | Flexible shaft assy with pin engaging “J” shape slot |
| 128 | | |
| 129 | Grasping means | Flexible shaft assy with threaded distal end of grasping means |
| 130 | Grasping means | Flexible shaft assy with no threads and no pin |
| 131 | Shaft middle portion | Shaft middle portion of all flexible shaft assembly |
| 132 | | |
| 133 | Distal end | Distal end of all flexible shaft assembly |
| 134 | | |
| 135 | Proximal end | Proximal end of all flexible shaft assy |
| 136 | | |
| 137 | Penetrating tip | Penetrating tip of flexible shaft 115 |
| 138 | Suture thread end | |
| 139 | | |
| 140 | Opposite suture thread end | |
| 141 | Suture thread | |
| 142 | “J” slot | Of guided tube for engaging with graspin means of flexible shaft |
| 143 | External thread | Threaded shank of grasping means |
| 144 | Shank | Smooth Shank of all smooth shank grasping means of flexible shaft assy |
| 145 | Distal end | Distal end of grasping means of all flexible shaft |
| 146 | Pin | Pin to lock griping means of flexible shaft assembly with guided tube |
| 147 | Shank | Shank of detachable taper tube |
| 148 | Split shank | Split shank to snap penetrator into guided tube |
| 149 | Large proximal end | Of Detachable Taper Tube |
| 150 | Outside perimeter | Of detachable taper tube |
| 151 | Small distal end | Small distal end of taper tube |
| 152 | Guided tube | with “J” Slot for locking engagement means |
| 153 | Opening | Opening at distal end of taper tube |
| 154 | Passage | Passage through the taper tube |
| 155 | Internal thread | Internal thread at proximal end of 103 guided tube |
| 156 | Internal thread | Internal threads in handle portion |
| 157 | Shoulder | Shoulder af taper tube |
| 158 | Shoulder | Shoulder of all penetrators |
| 159 | Rim | Rim at distal end of all guided tubes |
| 160 | | |
| 161 | Opening | For suture thread holder version |
| 162 | Hook | For suture thread holder version |
| 163 | Bend | Bend of all guided tubes |
| 164 | Cavity | For detachable penetrator with cavity |
| 165 | Stab wound incision | |
| 166 | Retractor | |
| 167 | Foley Catheter | |
| 168 | Anesthesia Syringe | |
| 169 | Lower abdomen | |
| 170 | | |
| 171 | Pubic Symphsis | |
| 172 | | |
| 173 | Urethra | |
| 174 | | |
| 175 | Blader neck | |
| 176 | | |
| 177 | Skin markings | |
| 178 | Anterior Vagina | |
| 179 | Vagina Opening | |
| 180 | Vagina mucosa | |
| 181 | | |
| 182 | | |
| 183 | | |
| 184 | | |
| 185 | Endopelvic Fascia | |
| 186 | | |
| 187 | Anterior Rectus Fascia | |
| 188 | | |
| 189 | Vaginal incision | |
| 190 | | |
| 191 | Sling material | |
| 192 | | |
| 193 | Tissue | |
| 194 | | |
| 195 | Tissue opening | |
| 196 | | |
| 197 | Detachable penetrator | Detachable penetrator with dimple |
| 198 | Detachable penetrator | Detachable penetrator with cavity and smooth shank |
| 199 | Penetrating tip | Penetrating tip of all penetrators |
| 200 | | |
| 201 | Proximal end | Proximal end of all penetrators except the one with the cavity |
| 202 | Large portion | Large portion of all penetrators except the one with the cavity |
| 203 | Split | Split for penetrator to snap into guided tube |
| 204 | Counterbore | Counterbore for penetrator with internal thread |
| 205 | Legs | Legs for penetrator to snap into guided tube |
| 206 | Proximal end | Proximal end of guided tube plain, without threads or “J” slot |
| 207 | Dimple | Dimple for penetrator to snap into guided tube |
| 208 | Taper ends | Taper ends to help penetrator to snap into guided tube |
| 209 | Side hole | Side hole in guided tube to snap detachable penetrator |
| 210 | Rim | Rim at proximal end Without internal threads for side hole version |
| 211 | Distal end | Distal end of all penetrators |
| 212 | Shoulder | Shoulder of Grasping means for flexible shaft assy without engagement means |
| 213 | Instrument | Instrument with Detachable penetrator with internal swage groove |
| 214 | Internal swage groove | Internal swage groove in guided tube to snap detachable penetrator |
| 215 | Guided tube | With internal swage groove at distal end |
| 216 | Instrument | Instrument with Detachable penetrator with cavity |
| 217 | Instrument | Instrument with Detachable penetrator with counterbore |
| 218 | Instrument | Instrument with Detachable penetrator with external threads |
| 219 | Guided tube | Plain guide tube for treaded distal end flexible shaft |
| 220 | Guided tube | Internal thread at guide tube distal end |
| 221 | Flexible shaft assembly | Shaft assy with treaded distal end of shaft |
| 222 | Instrument | Instrument with “J” engagement |
| 223 | External thread | External threads of distal end of flexible shaft |
| 224 | Internal thread | Threaded distal end of guided tube |
| 225 | Detachable penetrator | Detachable penetrator with internal thread |
| 226 | Detachable penetrator | Detachable penetrator with external thread |
| 227 | Shank | of detachable penetrator with internal thread & the one with a cavity |
| 228 | External thread | For detachable penetrator with external thread |
| 229 | Internal thread | Internal thread of detachable penetrator with internal thread |

| | | Instrument | Instrument | Instrument | Instrument | | |
|---|---|---|---|---|---|---|---|
| | | with | with | with | with | | Instrument |
| | | Detachable | Detachable | Detachable | Detachable | | with |
| | Instrument | penetrator | penetrator | penetrator | penetrator | Instrument | Detachable |
| | with Taper | with side hole | with swage | with external | with | with “J” | penetrator |
| | tube | tube | groove | threads | counterbore | engagement | with cavity |
| | 101 | 102 | 213 | 218 | 217 | 222 | 216 |
| Guide tube | 103 | 104 | 215 | 220 | 219 | 152 | 103 |
| flexible shaft assembly | 115 | 116 | 116 | 116 | 221 | 120 | 115 |
| Taper tube | 119 | | | | | 119 | |
| Penetrator | | 197 | 197 | 226 | 225 | | 198 |

| | Internal | | | | Plain guide | | |
|---|---|---|---|---|---|---|---|
| | thread | | | | tube for | Internal | |
| | guide | | | With | treaded | threads at | Internal |
| | tube at | “J” Slot | Side hole | swage | distal end | proximal | thread at |
| | proximal | locking | at distal | groove at | flexible | end of | guide tube |
| | end | means | end | distal end | shaft | handle assy | distal end |
| | 103 | 152 | 104 | 215 | 219 | 122 | 220 |
| Distal end | 105 | 105 | 105 | 105 | 105 | 105 | 105 |
| Proximal end | 107 | | | | | | |
| Outside perimeter | 150 | | | | | | |
| Stem | 109 | 109 | 109 | 109 | 109 | 109 | 109 |
| Distal portion | 111 | 111 | 111 | 111 | 111 | 111 | 111 |
| internal thread at proximal end | 155 | | | | | | |
| Rim at distal end | 159 | 159 | 159 | 159 | 159 | 159 | 159 |
| Bend | 163 | 163 | 163 | 163 | 163 | 163 | 163 |
| Handle assembly | 113 | 113 | 113 | 113 | 113 | 113 | 113 |
| Handle portion of guide tube | 123 | 123 | 123 | 123 | 123 | 123 | 123 |
| Proximal end of handle portion | 124 | 124 | 124 | 124 | 124 | 124 | 124 |
| Central bore of handle portion | 125 | 125 | 125 | 125 | 125 | 125 | 125 |
| Proximal end | | 106 | | | | | |
| “J” slot | | 142 | | | | | |
| Rim at proximal end Without internal threads | | | 210 | 210 | 210 | | |
| Proximal end of guide tube plain | | | 206 | 206 | 206 | | |
| Side hole | | | 209 | | | | |
| Swage groove | | | | 214 | | | |
| Threaded handle portion | | | | | | 156 | |
| Threaded didtal end of tube | | | | | | | 224 |

| | Shaft assy | | | |
|---|---|---|---|---|
| | with threaded | Shaft assy | Shaft assy | Shaft assy |
| | distal end of | with treaded | with pin | plain |
| | grasping | distal end of | engaging “J” | (nothreads, |
| | means | shaft | shape slot | no pin |
| | 115 | 221 | 118 | 116 |
| Grasping means | 129 | 130 | 127 | 130 |
| Suture thread holder | 117 | 117 | 117 | 117 |
| Opening | 161 | | | |
| Threaded shank of grasping means | 143 | | | |
| Distal end of grasping means | 145 | 145 | 145 | 145 |
| Proximal end of flex shaft 121 | 135 | 135 | 135 | 135 |
| Flexible shaft | 121 | 121 | 121 | 121 |
| Flex shaft middle portion | 131 | 131 | 131 | 131 |
| Suture thread holder | 117 | 117 | | 117 |
| Distal end of flexible shaft assembly | 133 | 133 | 133 | 133 |
| Penetrating tip of flexible shaft | 137 | | | |
| Shoulder of Grasping means | | | | 212 |
| Smooth Shank of grasping means | | 144 | 144 | 144 |
| External threads of distal end of flexible shaft | | 223 | | |
| Pin | | | 146 | |

| | | | Detachable | Detachable | |
|---|---|---|---|---|---|
| | | Detachable | penetrator | penetrator | Detachable |
| | Detachable | penetrator | with internal | with external | penetrator |
| | Taper Tube | with dimple | thread | thread | with cavity |
| | 119 | 197 | 225 | 226 | 198 |
| Large proximal end | 149 | | | | |
| Shank of taper tube | 147 | | | | |
| Small distal end | 151 | | | | |
| Opening at distal end | 153 | | | | |
| Passage through the tube | 154 | | | | |
| Shoulder of taper tube | 157 | | | | |
| Proximal end of penetrator | | 201 | 201 | 201 | |
| Split | | 203 | | | |
| Legs | | 205 | | | |
| Dimple | | 207 | | | |
| Taper ends | | 208 | | | |
| Shank, Split | | 148 | | | |
| Shoulder of penetrator | | 158 | 158 | 158 | 158 |
| Penetrating tip | | 199 | 199 | 199 | 199 |
| Large portion of penetrator | | 202 | 202 | 202 | |
| Distal end of penetrator | | 211 | 211 | 211 | 211 |
| internal thread | | | 229 | | |
| Counterbore | | | 204 | | |
| Shank | | | 227 | | 227 |
| External thread | | | | 228 | |
| Cavity | | | | | 164 |

Having thus described our invention, what we claim is new and desire to secure by United States Letter Patent is:

1. An instrument which aids inserting a Pubovaginal Sling to correct stress urinary incontinence (SUI) in women, comprising
a substantially rigid guide tube having opposite open ends which are distal and proximal relative to a person using the instrument, said guide tube having a stem portion and a distal end portion extending generally laterally with respect to the stem portion and with locking engagement means at the proximal end,
a handle on the stem portion of the guide tube generally adjacent the proximal end of the guide tube,
a stiff but flexible shaft inside the guide tube having a distal end part movable distally relative to the guide tube,
a detachable tapered tube having open ends with the large proximal end partially inserted inside the distal end of the rigid guide tube with means to prevent the further penetration of the detachable tapered tube into the guide tube and with the smaller distal end of the detachable tapered tube with an opening with just enough clearance to allow the exiting of said flexible shaft,
a suture thread holder with a penetrating tip is provided on the distal end part of the shaft extending through and beyond the open distal end of the detachable tapered tube,
said flexible shaft with grasping means at the proximal end externally to the guide tube with locking engagement means for engaging with the guide tube's proximal end locking engagement means during the instrument insertion,
said shaft being manually movable longitudinally relative to the guide tube when the guide tube locking means and the flexible shaft locking means are not engaged, to move the distal end part of the shaft from an retracted position in which the suture thread holder is adjacent to the open distal end of detachable tapered tube, to a extended position in which the suture thread holder is farther away from the distal end of the guide tube, the distal end portion of the guide tube and the detachable tapered tube being configured to guide the distal end part of the shaft in place and the suture thread holder thereon along a substantially straight path extending generally laterally at an angle of not less than about ninety degrees with respect to the stem portion of the guide tube with the bend close to the distal end of said guide tube forcing the side of said flexible shaft to develop a normal force against the internal wall of the detachable tapered tube as the stiff flexible shaft tends to go in a straight line, said normal force creates enough friction to keep said detachable tapered tube from separating from said guide tube.

2. An instrument as set forth in claim 1 wherein said suture thread holder comprises an opening formed in the distal end part of the shaft.

3. A suturing instrument as set forth in claim 2 wherein said opening is an eye.

4. A suturing instrument as set forth in claim 1 wherein the shaft is a one-piece plastic shaft including the grasping means, the threaded portion, the penetrating tip and a suture thread holder.

5. An instrument which aids inserting a Pubovaginal Sling to correct stress urinary incontinence (SUI) in women, comprising
a substantially rigid guide tube having opposite open ends which are distal and proximal relative to a person using the instrument, said guide tube having a stem portion and a distal end portion extending generally laterally with respect to the stem portion and with an aperture on the side of said guide tube at the extreme of the distal end,
a handle on the stem portion of the guide tube generally adjacent the proximal end of the guide tube,
a detachable penetrator having opposite ends with a shank at the proximal end of said detachable penetrator constructed so it is capable of being inserted inside the distal end of said rigid guide tube, with means to be kept in place in said guide tube by engaging a resilient protrusion on the side of said shank into the aperture on the side of said guide tube and with a penetrating tip at the distal end of said detachable penetrator,
a stiff but flexible shaft inside said guide tube having a distal end part movable distally relative to the guide tube when the detachable penetrator is not attached to the guide tube,
a suture thread holder on the distal end part of the shaft and griping means at the proximal end of the flexible shaft externally to the guide tube,
said shaft, when the detachable penetrator is not connected to the guide tube, being manually movable longitudinally relative to the guide tube to move the distal end part of the shaft from an retracted position in which the suture thread holder is adjacent to the open distal end of the guide tube, to a extended position in which the suture thread holder is farther away from the distal end of the guide tube, the distal end portion of the guide tube being configured to guide the distal end part of the shaft and the suture thread holder thereon along a substantially straight path extending generally laterally at an angle of not less than about ninety degrees with respect to the stem portion of the guide tube.

6. An instrument as set forth in claim 5 wherein said suture thread holder comprises an opening formed in the distal end part of the shaft.

7. A suturing instrument as set forth in claim 6 wherein said opening is an eye.

8. A suturing instrument as set forth in claim 5 wherein the shaft is a one-piece plastic shaft including the grasping means and a suture thread holder.

9. An instrument which aids inserting a Pubovaginal Sling to correct stress urinary incontinence (SUI) in women, comprising
a substantially rigid guide tube having opposite open ends which are distal and proximal relative to a person using the instrument, said guide tube having a stem portion and a distal end portion extending generally laterally with respect to the stem portion and with an internal swage groove 360 degrees internally very close to the distal end,
a handle on the stem portion of the guide tube generally adjacent the proximal end of the guide tube,
a detachable penetrator having opposite ends with a shank at the proximal end of said detachable penetrator constructed so it is capable of being inserted inside the distal end of said rigid guide tube, with means to be kept in place in said guide tube by engaging a resilient protrusion on the side of said shank into the internal swage groove of said guide tube and with a penetrating tip at the distal end of said detachable penetrator,
a stiff but flexible shaft inside said guide tube having a distal end part movable distally relative to the guide tube when the detachable penetrator is not attached to the guide tube,
a suture thread holder on the distal end part of the shaft and griping means at the proximal end of the flexible shaft externally to the guide tube,
said shaft, when the detachable penetrator is not connected to the guide tube, being manually movable longitudinally relative to the guide tube to move the distal end part of the shaft from a retracted position in which the suture thread holder is adjacent to the open distal end of the guide tube, to a extended position in which the suture thread holder is farther away from the distal end of the guide tube, the distal end portion of the guide tube being configured to guide the distal end part of the shaft and the suture thread holder thereon along a substantially straight path extending generally laterally at an angle of not less than about ninety degrees with respect to the stem portion of the guide tube.

10. An instrument as set forth in claim 9 wherein said suture thread holder comprises an opening formed in the distal end part of the shaft.

11. A suturing instrument as set forth in claim 10 wherein said opening is an eye.

12. A suturing instrument as set forth in claim 9 wherein the shaft is a one-piece plastic shaft including the grasping means and a suture thread holder.

13. An instrument which aids inserting a Pubovaginal Sling to correct stress urinary incontinence (SUI) in women, comprising
a substantially rigid guide tube having opposite open ends which are distal and proximal relative to a person using the instrument, said guide tube having a stem portion and a distal end portion extending generally laterally with respect to the stem portion;
a handle on the stem portion of the guide tube generally adjacent the proximal end of the guide tube,
a detachable penetrator having opposite ends with a shank at the proximal end of said detachable penetrator constructed so it is capable of being inserted inside the distal end of said rigid guide tube, with a counterbore threaded hole at the proximal end and with a penetrating tip at the distal end of said detachable penetrator,
a stiff but flexible shaft inside said guide tube having a external threaded distal end part movable distally relative to the guide tube when the detachable penetrator is not attached to the guide tube, said threaded distal end part for engagement with the internal thread of said counterbore threaded hole at the proximal end of the said detachable penetrator,
a suture thread holder on the distal end part of the shaft and griping means at the proximal end of the flexible shaft externally to the guide tube,
said shaft, when the detachable penetrator is not connected to the guide tube, being manually movable longitudinally relative to the guide tube to move the distal end part of the shaft from an retracted position in which the suture thread holder is adjacent to the open distal end of the guide tube, to an extended position in which the suture thread holder is farther away from the distal end of the guide tube, the distal end portion of the guide tube being configured to guide the distal end part of the shaft and the suture thread holder thereon along a substantially straight path extending generally laterally at an angle of not less than about ninety degrees with respect to the stem portion of the guide tube.

14. An instrument as set forth in claim 13 wherein said suture thread holder comprises an opening formed in the distal end part of the shaft.

15. A suturing instrument as set forth in claim 14 wherein said opening is an eye.

16. A suturing instrument as set forth in claim 13 wherein the shaft is a one-piece plastic shaft including the grasping means and a suture thread holder.

17. An instrument which aids inserting a Pubovaginal Sling to correct stress urinary incontinence (SUI) in women, comprising
a substantially rigid guide tube having opposite open ends which are distal and proximal relative to a person using the instrument, said guide tube having a stem portion and a distal end portion extending generally laterally with respect to the stem portion and with internal threads at the distal end,
a handle on the stem portion of the guide tube generally adjacent the proximal end of the guide tube,
a detachable penetrator having opposite ends with external threads at the proximal end of said detachable penetrator constructed so it is capable of being engaged with the internal threads of the distal end of said guide tube and with a penetrating tip at the distal end of said detachable penetrator,
a stiff but flexible shaft inside said guide tube having a distal end part movable distally relative to the guide tube when the detachable penetrator is not attached to the guide tube,
a suture thread holder on the distal end part of the shaft and griping means at the proximal end of the flexible shaft externally to the guide tube,
said shaft, when the detachable penetrator is not connected to the guide tube, being manually movable longitudinally relative to the guide tube to move the distal end part of the shaft from an retracted position in which the suture thread holder is adjacent to the open distal end of the guide tube, to a extended position in which the suture thread holder is farther away from the distal end of the guide tube, the distal end portion of the guide tube being configured to guide the distal end part of the shaft and the suture thread holder thereon along a substantially straight path extending generally laterally at an angle of not less than about ninety degrees with respect to the stem portion of the guide tube.

18. An instrument as set forth in claim 17 wherein said suture thread holder comprises an opening formed in the distal end part of the shaft.

19. A suturing instrument as set forth in claim 18 wherein said opening is an eye.

20. A suturing instrument as set forth in claim 17 wherein the shaft is a one-piece plastic shaft including the grasping means and a suture thread holder.

21. An instrument which aids inserting a Pubovaginal Sling to correct stress urinary incontinence (SUI) in women, comprising
a substantially rigid guide tube having opposite open ends which are distal and proximal relative to a person using the instrument, said guide tube having a stem portion and a distal end portion extending generally laterally with respect to the stem portion and with locking engagement means at the proximal end,
a handle on the stem portion of the guide tube generally adjacent the proximal end of the guide tube,
a detachable penetrator having opposite ends with a shank at the proximal end of said detachable penetrator constructed so it is capable of being inserted inside the distal end of said rigid guide tube and with a penetrating tip at the distal end of said detachable penetrator,
a stiff but flexible shaft inside said guide tube having a distal end part movable distally relative to the guide tube,
a suture thread holder on the distal end part of the shaft and griping means at the proximal end of the flexible shaft externally to said guide tube and with locking engagement means at the distal end of said griping means for engaging with the locking engagement means at the proximal end of said guide tube during the instrument insertion during the procedure,
a dead end cavity at the distal end of said penetrator to make room for the distal end of said flexible shaft when said flexible shaft and said guide tube threads are engaged,
said shaft, being manually movable longitudinally relative to the guide tube to move the distal end part of the shaft from a retracted position in which the suture thread holder is adjacent to the open distal end of the guide tube, to a extended position in which the suture thread holder is farther away from the distal end of the guide tube, the distal end portion of the guide tube being configured to guide the distal end part of the shaft and the suture thread holder thereon along a substantially straight path extending generally laterally an angle of not less than about ninety degrees with respect to the stem portion of the guide tube with the bend close to the distal end of said guide tube forcing the side of said flexible shaft to develop a normal force against the internal wall of the dead end cavity as the stiff flexible shaft tends to go in a straight line, said normal force creating enough friction to keep said detachable penetrator from separating from said guide tube.

22. An instrument as set forth in claim 21 wherein said suture thread holder comprises an opening formed in the distal end part of the shaft.

23. A suturing instrument as set forth in claim 22 wherein said opening is an eye.

24. A suturing instrument as set forth in claim 21 wherein the shaft is a one-piece plastic shaft including the grasping means and a suture thread holder.

25. A pubovaginal sling implantation procedure to correct stress urinary incontinence (SUI) in women, comprising
of placing a standard retractor on the lower vaginal opening to facilitate the procedure,
making two small stab-wound incisions about an inch apart on the center of the female's lower abdomen just above the pubic symphysis,
separating the subcutaneous tissue to the anterior rectus fascia with blunt dissection,
placing a Foley catheter in the urethra to help identify the urethra and the bladder neck by palpation,
making a two-centimeter skin marking in the anterior vagina along the urethra,
infiltrating with local anesthetic containing epinephrine, then an incision is made in the anterior vagina following the skin markings,
the vagina mucosa is separated from the urethra with combination of sharp and blunt dissection for a distance of about 1 to 2 centimeters with enough space created to allow the palpation of the endopelvic fascia to the lateral urethra with the surgeon's finger,
the instrument of claim 1 is assembled, then with one hand, the person performing the procedure grasps the instrument of claim 1 by the handle assembly, inserting the distal end of the flexible shaft assembly into one of the suprapubic stab-wound incisions, thrusting the instrument while the penetrating tip of the suture thread holder perforates through the anterior rectus fascia, and then sliding along the posterior surface of the pubic symphysis,
with the surgeon's finger on the endopelvic fascia to the lateral urethra, the introducing penetrating tip should be palpable by the surgeon's finger and the penetrating tip is guided into the vaginal incision through the endopelvic fascia,
the thrust of the instrument is continued until the detachable tapered tube clears the vaginal incision, the flexible shaft assembly is unscrewed and partially withdrawn from the guide tube until the flexible shaft assembly clears the detachable tapered tube which will release the pressure of the side of the flexible shaft against the inner wall of the detachable tapered tube,
then the detachable tapered tube is removed from the guide tube by manually pulling the detachable tapered tube away from the guide tube, now the flexible shaft assembly is thrust again toward the distal end of the guide tube, until the suture thread holder is exposed at the distal end of the guide tube, next, a piece of standard sling material is used and the sling material is cut into approximately one-centimeter by ten centimeters in size,
a long piece of suture thread is then inserted into one end of the sling material, both ends of the suture thread is then inserted and passed through the opening of distal end portion of the flexible shaft assembly, then both ends of the suture thread are pulled until the sling material is adjacent to the distal end of the flexible shaft assembly and the flexible shaft assembly is withdrawn from the guide tube and retracted further into the guide tube bringing along a section of the sling material and a section of the suture thread into the guide tube,
after the desired penetration of the sling material is obtained inside the guide tube, the flexible shaft assembly is withdrawn further while maintaining the tension on the exposed portion of the sling material, then finally the flexible shaft assembly is pulled away from the guide tube until both ends of the suture thread exits the guide tube, the ends of suture thread are then pulled through the suture thread holder of flexible shaft assembly,
while holding with the fingers the portion of the sling material that is exposed, the guide tube is withdrawn leaving the sling material in the desired location inside the tissue and with the sling material being compressed by the contracting tissue and the guide tube is continued to be withdrawn until the guide tube is completely away from the abdominal stab-wound incision and the suture thread exits the guide tube with the suture thread being exposed from the abdominal stab-wound incision,
the detachable tapered tube is again connected inside the distal end of the guide tube with the flexible shaft assembly protruding through the detachable tapered tube and the external threads of the flexible shaft assembly and the internal threads of guide tube locks into place, then the instrument is inserted the same way as previously explained, inside the other abdominal stab-wound incision, this time the instrument is guided on the other side of the urethra,
after the detachable tapered tube is removed, another suture thread is inserted through the exposed end of the sling material, then both suture thread ends of the suture thread are inserted and passed through the suture thread holder and both suture ends of suture thread are pulled until the sling material is adjacent to the suture thread holder of the flexible shaft, then the flexible shaft assembly is withdrawn from the guide tube bringing along this section of the sling material,
as the flexible shaft assembly is being withdrawn, the detachable tapered tube is placed between the looped sling material and the urethra, being used as a spacer to limit the travel of the sling material as the flexible shaft assembly continues to be withdrawn from the guide tube, bringing the sling material further into the guide tube thus preventing the excessive pressure of the sling material on the bladder neck, avoiding the risk of postop urinary retention or erosion of the sling material into the urethra,
after the sling material is tight around the detachable tapered tube, the flexible shaft assembly continues to be withdrawn away from the guide tube bringing the suture thread outside the proximal end of the guide tube, and the flexible shaft assembly continues to be pulled away from the guide tube until the suture thread is released from the suture thread holder of the flexible shaft assembly leaving the suture thread exposed at the proximal end of the guide tube,
the guide tube is then pulled away from the tissue leaving the sling material in place, then the detachable tapered tube is now removed from the area leaving the entire sling material implanted,
now one end of an exposed suture thread is pulled until the entire suture thread is away from the stab-wound incisions and the same process is repeated with the other suture thread protruding from the other stab-wound incisions, the vaginal incision is closed with absorbable suture thread and the lower abdominal stab-wound incisions normally only require Steri-Strips, the Foley catheter and the retractor are now removed to complete the procedure.

26. An instrument which aids inserting a Pubovaginal Sling to correct stress urinary incontinence (SUI) in women, comprising
a substantially rigid guide tube having opposite open ends which are distal and proximal relative to a person using the instrument, said guide tube having a stem portion and a distal end portion extending generally laterally with respect to the stem portion,
a handle on the stem portion of the guide tube generally adjacent the proximal end of the guide tube,
a detachable penetrator having opposite ends with a shank at the proximal end and with a penetrating tip at the distal end, constructed so it is capable of being inserted inside the distal end of said rigid guide tube and with engagement means to keep said penetrator and said rigid guide tube together,
a stiff but flexible shaft inside said guide tube having a distal end part movable distally relative to the guide tube when the detachable penetrator is not attached to the guide tube,
a suture thread holder on the distal end part of the shaft and griping means at the proximal end of the flexible shaft externally to the guide tube,
said shaft, when the detachable penetrator is not connected to the guide tube, being manually movable longitudinally relative to the guide tube to move the distal end part of the shaft from a retracted position in which the suture thread holder is adjacent to the open distal end of the guide tube, to an extended position in which the suture thread holder is farther away from the distal end of the guide tube, the distal end portion of the guide tube being configured to guide the distal end part of the shaft and the suture thread holder thereon along a substantially straight path extending generally laterally at an angle of not less than about ninety degrees with respect to the stem portion of the guide tube.
